(19) United States
(12) Patent Application Publication
(10) Pub. No.: US 2020/0147164 A1
(43) Pub. Date: May 14, 2020

(54) "View nutrition" Take4B4 cold symptoms (Natural vitamin C), soothing triple gel thirst quencher, powdered banana leaves (Intense fiber), UTI (Urinary tract infection) buster

(71) Applicant: Joe Ann Aiken, Sumter, SC (US)

(72) Inventor: Joe Ann Aiken, Sumter, SC (US)

(21) Appl. No.: 16/350,398

(22) Filed: Nov. 13, 2018

(51) Int. Cl. A61K 36/752; A61K 36/88; A61P 13/02; A23L 33/105; A61K 9/00; A61K 31/375; A23L 19/00; A61K 36/81; A23L 33/22; A61K 36/9068; A23L 2/06; A61K 36/45; A61P 11/00; A23L 2/52; A23L 2/60; A23L 29/262; A61K 36/73

(52) U.S. Cl. CPC A61K 36/752; A23V 2002/00; A61P 13/02; A23L 33/105; A61K 9/0095; A61K 31/375; A23L 19/09; A61K 36/81; A23L 33/22; A61K 36/9068; A23L 2/06; A61K 36/45; A61P 11/00; A23L 2/52; A23L 2/60; A23L 29/262; A61K 36/73; A61K 36/88

(57) ABSTRACT

The Take4B4 drink contains phytochemicals which is found in onions along with Vitamin C to help improve immunity. Onions is said to help reduce inflammation and heal infections. Raw onions lower the production of bad cholesterol (LDL). A powerful compound called quercetin in onions is known to play a significant role in preventing cancer. Lemons—the acid in lemons helps to neutralize free radicals linked to aging and most types of disease. The citric acid in lemon juice helps to dissolve gallstones, calcium deposits, and kidney stones. Lemons are rich in vitamin C and flavonoids that work against infections like the flu and colds.

Soothing Triple Gel Thirst Quencher, the Aloe Vera contains Vitamin C, and other Vitamin and minerals. Okra contains Vitamin C and calcium. Lemons naturally have Vitamin C, and the Nopal Cactus contains anti-inflammatory agents. Nopal Cactus—said to control diabetes, is an anti-inflammatory agent, help improve sleep. Okra contains potassium, vitamin B, vitamin C, folic acid, and calcium.

Powdered Banana Leaves (Intense Fiber)—Plant fiber that can be used for shakes and smoothies, or baking. The chlorophyll in the leaves prevents intestinal ulcer and skin disease and it cleanses the blood.

UTI (Urinary Tract Infection—This is a natural sugar-free drink with healing properties. Cranberries may be useful as prevention against urinary tract infections, stomach cancer, and heart disease. Garlic is a plant in the Allium (onion) family closely related to onions, shallots, leeks, chives, and scallions; these can be substituted in the recipe to replace the garlic.

### CROSS REFERENCE TO RELATED APPLICATION

This application claims the benefits of U.S. provisional application No. 62/707,082, filed Oct. 23, 2017, the disclosure of which is incorporated herein by reference.

### STATEMENT REGARDING FEDERALLY SPONSORED RESEARCH OR DEVELOPMENT

“Not Applicable”

### BACKGROUND OF INVENTION

Garlic is a plant in the Allium (onion) family closely related to onions, shallots, leeks, chives, and scallions; these can be substituted in the recipe to replace the garlic, giving the product some of the same nutritious benefits as the garlic. The garlic has powerful medicinal benefits with the active ingredient of Allicin. Onions contains chromium, which assists in regulating blood sugar. The phytochemicals in onions along with their Vitamin C help improve immunity. Onions is said to help reduce inflammation and heal infections. Raw onions lower the production of bad cholesterol (LDL). A powerful compound called quercetin in onions is known to play a significant role in preventing cancer. Bell Peppers contain plenty of vitamin C, which powers up your immune system and keeps skin youthful. Red bell peppers contain several phytochemicals and carotenoids, particularly beta-carotene, which lavish you with antioxidant and anti-inflammatory benefits. The capsaicin in bell peppers has multiple health benefits. Studies show that it reduces bad cholesterol, controls diabetes, and it brings relief from pain and eases inflammation. Lemons—the acid in lemons helps to neutralize free radicals linked to aging and most types of disease. The citric acid in lemon juice helps to dissolve gallstones, calcium deposits, and kidney stones. Lemons are rich in vitamin C and flavonoids that work against infections like the flu and colds. www.care2.com/greenliving

Nopal Cactus—said to control diabetes, is an anti-inflammatory agent, help improve sleep. Build strong bones, help prevent cancer. www.organifacts.net

Okra contains potassium, vitamin B, vitamin C, folic acid, and calcium. Okra has been suggested to help manage blood sugar in cases of type 1, type 2, and gestational diabetes.
Aloe Vera contains many vitamins including A, C, E, folic acid, and choline. Aloe Vera is also one of the few plants that contains vitamin B12. Some of the 20 minerals found in Aloe Vera include: calcium, magnesium, zinc, chromium, selenium, sodium, iron, potassium, copper, manganese.
Banana leaves in addition to their value as food wrappers for cooking, banana leaves contain large amounts of polyphenols such as epigallocatechin gallate, or EGCG, also found in green tea. www.amazetify.com

Cranberries may be useful as prevention against urinary tract infections, stomach cancer, and heart disease. www.Whfoods.ord

Blueberries are rich in proanthocyanidin, contributing to blueberry benefits that include fighting cancer, losing weight and glowing, young skin. Blueberries are also rich in vitamin C, vitamin K, manganese and other antioxidants that lead to it numerous health benefits. www.draxe.com
Strawberries are an excellent source of vitamins C and K as well as providing a good dose of fiber, folic acid, manganese and potassium. www.bbcgoodfood.com
Ginger the root or stem known as the rhizome is packed with phenolic compounds that helps improve your digestive system, when consumed regularly. It is also able to reduce the feeling of nausea, and bolster your body's immune system to combat common cold, flu and similar problems. “If you're feeling bad, you can count on ginger to perk you up.” www.dailynaturalremedies.com

Banana leaves The wrapped banana leaves that form the plant's stem contain starch, which is extracted through a fermentation or cooking process. Powdered Banana Leaves (Intense Fiber). Benefits of eating banana leaves: The chlorophyll in the leaves prevents intestinal ulcer and skin disease.

### SUMMARY OF INVENTION

Take 4B4 Cold Symptoms with natural Vitamin C can be used to ward off the common cold. This invention makes it easy to consume Vitamin C in the natural state.

The banana leaves powder will provide another way to add fiber to the diet. This is the first time that banana leaves are introduced in form of powder, making it an easy way to get extra fiber in our diets.

The Soothing Triple Gel Thirst Quencher, is a natural soothing way to quench your thirst on a summer day. This is an easy way to get the gel from three healthy plants. This soothing drink can be a refreshing cool down on a hot summer day.

The UTI Buster can be a natural alternative to medication for treating urinary tract infections. All natural shake that can be beneficial to the healing process of Urinary Tract Infections.

### DETAILED DESCRIPTION OF DRAWINGS

Not Applicable

### DETAILED DESCRIPTION OF THE INVENTION

Take4B4 Cold Symptom is a natural drink is loaded with Vitamin C. Vitamin C can help keep the common cold at bay. This natural drink can be consumed anytime. The citrus juice, bell peppers, onions, and garlic all have natural Vitamin C. Vitamin C is said to help with the common cold. The phytochemicals in onions along with their Vitamin C help improve immunity. Onions is said to help reduce inflammation and heal infections. Raw onions lower the production of bad cholesterol (LDL). A powerful compound called quercetin in onions is known to play a significant role in preventing cancer. Lemons—the acid in lemons helps to neutralize free radicals linked to aging and most types of disease. The citric acid in lemon juice helps to dissolve gallstones, calcium deposits, and kidney stones. Lemons are rich in vitamin C and flavonoids that work against infections like the flu and colds. www.care2.com/greenliving

Soothing Triple Gel Thirst Quencher, the Aloe Vera contains Vitamin C, and other Vitamin and minerals. Okra contains Vitamin C and calcium. Lemons naturally have Vitamin C, and the Nopal Cactus contains anti-inflammatory agents. Nopal Cactus—said to control diabetes, is an anti-inflammatory agent, help improve sleep. Build strong bones, help prevent cancer. www.organifacts.net Okra contains potassium, vitamin B, vitamin C, folic acid, and calcium. Okra has been suggested to help manage blood sugar in cases of type 1, type 2, and gestational diabetes. Aloe Vera contains many vitamins including A, C, E, folic acid, choline, B1, B2, B3 (niacin), B6. Aloe Vera is also one of the few plants that contains vitamin B12. Some of the 20 minerals found in Aloe Vera include: calcium, magnesium, zinc, chromium, selenium, sodium, iron, potassium, copper, manganese.

Powdered Banana Leaves (Intense Fiber). Benefits of eating banana leaves: 1. The chlorophyll in the leaves prevents intestinal ulcer and skin disease. 2. It cleanses the blood. 3. It stimulates appetite and help digest food. 4. It soothes the mucous lining in the stomach and cures ulcer. It is a good remedy for kidney stones and other bladder related diseases. It boosts immune system. www.curejoy.com

Urinary Tract Infection (UTI) Buster—Is a natural sugar free beverage to help with the symptoms of UTI. Cranberries may be useful as prevention against urinary tract infections, stomach cancer, and heart disease. www.Whfoods.ord Blueberries are rich in proanthocyanidin, contributing to blueberry benefits that include fighting cancer, losing weight and glowing, young skin. Blueberries are also rich in vitamin C, vitamin K, manganese and other antioxidants that lead to it numerous health benefits. www.draxe.com Strawberries are an excellent source of vitamins C and K as well as providing a good dose of fiber, folic acid, manganese and potassium. www.bbcgoodfood.com Ginger the root or stem known as the rhizome is packed with phenolic compounds that helps improve your digestive system, when consumed regularly. It is also able to reduce the feeling of nausea, and bolster your body's immune system to combat common cold, flu and similar problems. “If you're feeling bad, you can count on ginger to perk you up.” www.dailynaturalremedies.com

### Take4B4 Cold Symptoms (Natural Vitamin C)

¼ cup citrus juice (can use citrus fruit, meat, peel, skin)
½ cup chopped bell peppers (red, orange, yellow, green, sweet peppers)
¼ cup chopped onions (any variety of onions, any color)
1 garlic clove
½-1 gallon of water (flavored, carbonated)
2 tablespoon sweetener

### Steps to Making Take4B4:

Instructions: Bring water to a boil, add citrus juice or fruit, peppers, onion and garlic, let boil on high for about three (3) minutes, then let simmer on low for another 10-15 minutes. Stir in the sweetener and let cool enough to pour into a container. Note: can be consumed either warm or cold. If you don't want the pulp, just pour the liquid off the ingredients, if you want the pulp and liquid, simply pour the content into a blender and blend until smooth.
You can also blend all the raw ingredients together with the water and sweetener and let it sit in the refrigerator for 12-24 hours, and you can enjoy the nutrients from a raw state. Note: This is not just a flu season drink; drink it all year round to fight the cold and flu symptoms. Note: This product can also be packaged as a frozen smoothie pack minus the water.

### Soothing Triple Gel Thirst Quencher

¼ cup citrus juice 1 cup chopped cactus meat 1 cup chopped aloe leaf 1 gallon water (other liquids) 1 cup okra cut in small pieces container for the drink
Steps to making the Soothing Triple Gel Thirst Quencher:
Cut the needles off of the cactus and aloe leaves, and then cut them into small to medium pieces, cut the okra in 3-4 pieces. Pour the water into the container, add the above ingredients, refrigerate for 24-48 hours or longer, allowing the citrus acid to pull the gel from the cactus, aloe, and okra plants. Step 2: You can also put all the ingredients into a blender and blend, and enjoy the drink with pulp. Note: Can be packaged as a frozen smoothie pack minus the water.

### Powdered Banana Leaves (Intense Fiber)

### Steps to Powdering the Banana Leaves:

Banana leaves (as many as needed to make powder)
Rinse the banana leaves with cold water.
Dry them off with a clean towel or paper towel, to remove the excess water from the leaves.
Pull the leafy parts of the leaf from the stem.
Place the leaves on a baking sheet lined with parchment paper.
Place the pan in the oven at 350 degrees for approximately 20 minutes, then turn the oven down to 170 degrees, and let the leaves dry out completely (dehydrate).
Remove the pan from the oven and let cool completely.
Place the banana leaves into a blender and blend until powdered.
Pour the powder onto a piece of parchment paper and allow to cool.
Put in a dry airtight container or zip lock bag, until ready to use for baking, healthy shakes, or as a thickening agent for soups and stews just to name a few uses.

### UTI (Urinary Tract Infection) Buster

1 cup blueberries (fresh or frozen, no sugar added)
1 cup cranberries (fresh or frozen, no sugar added)
1 cup strawberries (fresh or frozen, no sugar added)
¼ cup chopped ginger (from ginger root or the jar)
2 tablespoons of citrus juice
2 garlic cloves
1 tablespoon Stevia (optional)
water (16-20 oz)
Steps to making the Urinary Tract Infection (UTI) Buster
Place all of the above ingredients into a blender and blend until smooth (will have pulp). Refrigerate the unused portion for later. This product can be bottled, canned, in a plastic container, or packaged as a frozen smoothie pack minus the water.

1. Take4B4 Cold Symptoms (Natural Vitamin C) comprising of:
a. Between 2-4 tablespoons citrus juice (can use citrus fruit, meat, peel, skin)
b. Between ¼-½ cup chopped bell peppers (red, orange, yellow, green, sweet peppers)
c. Between ¼-½ cup chopped onions (any variety of onions, any color)
d. Between 1-2 garlic clove
e. Between 1-2 quarts of water (flavored, carbonated)
f. Between 3-6 tablespoon sweetener
g. Instructions: Bring water to a boil; add citrus juice or ½ lemon, peppers, onion and garlic.
h. Boil on high for about three (3) minutes, then let simmer on low for another 10-15 min.
i. Stir in the sweetener and let cool enough to pour into a container. Note: Can be consumed either warm or cold. If you don't want the pulp, just pour the liquid off the ingredients, if you want the pulp and liquid, simply pour the content into a blender and blend until smooth.
j. You can also blend all the raw ingredients together with the water and sweetener and let it sit in the refrigerator for 12-24 hours, and you can enjoy the nutrients from a raw state. Note: This is not just a flu season drink; consume year round to fight the cold and flu symptoms. This product can also be packaged as a frozen smoothie pack minus the water.

2. Triple Gel Thirst Quencher

3. Powdered Banana Leaves (Intense Fiber) comprising of:
a. Banana leaves (as many as needed to make powder)
b. Rinse the banana leaves with cold water.
c. Dry them off with a clean towel or paper towel, to remove the excess water from the leaves.
d. Pull the leafy parts of the leaf from the stem.
e. Place the leaves on a baking sheet lined with parchment paper.
f Place the pan in the oven at 350 degrees for approximately 20 minutes.
g. Turn the oven down to 170 degrees, and let the leaves become completely dehydrated.
h. Remove the pan from the oven and let cool completely.
i. Place the banana leaves into a blender and blend until powdered.
j. Pour the powder onto a piece of dry parchment paper and allow to cool.
k. Put in a dry airtight container or zip lock bag, until ready to use for baking, healthy
l. shakes, or as a thickening agent for soups and stews.

4. UTI (Urinary Tract Infection) Buster comprising of:
a. Between ¾-1 cup blueberries (fresh or frozen, no sugar added)
b. Between ¾-1 cup cranberries (fresh or frozen, no sugar added)
c. Between ¾-1 cup strawberries (fresh or frozen, no sugar added)
d. 1-2 Tablespoons chopped ginger (from ginger root or the jar)
e. 2-3 tablespoons of citrus juice
f. 1-2 garlic cloves
g. 1 tablespoon Stevia (optional)
h. water (16-20 oz)
Place all of the above ingredients into a blender and blend until smooth (will have pulp). Refrigerate the unused portion for later. This product can be bottled, canned, in a plastic container, or packaged as a frozen smoothie pack minus the water.
